(19) United States
(12) Patent Application Publication
(10) Pub. No.: US 2015/0045859 A1
(43) Pub. Date: Feb. 12, 2015

(54) Ice Therapy System

(71) Applicant: Neil J. Kay, Rancho Palos Verdes, CA (US)

(72) Inventor: Neil J. Kay, Rancho Palos Verdes, CA (US)

(21) Appl. No.: 13/964,750

(22) Filed: Aug. 12, 2013

(51) Int. Cl. A61F 7/10

(52) U.S. Cl. CPC A61F 7/103; A61F 2007/0042
USPC 607/108; 607/114; 607/112

(57) ABSTRACT

A therapeutic ice bag shaped to cover a specific body part of a wearer. A timer is positioned on the bag to enable the user to determine when to remove the bag after a period of use.

### BACKGROUND OF THE INVENTION

1. Field of the Invention

The present invention relates to an ice pack designed to fit about a person's knee to effectuate the application of cold.

2. Description of the Prior Art

The prior art discloses numerous devices related to cyrotherapy i.e. using cooling as a therapeutic medical procedure to prevent swelling and pain in body parts that have been injured, such as a person's knee.

U.S. Pat. No. 7,704,273 to Morris et al discloses a therapeutic cold pack having a sleeve member adapted to accommodate a portion of the let of an individual. Attached to a surface of the therapeutic cold pack are one or more containment bags each defining an internal volume adapted to hold a therapeutic amount of a cold substance. Each containment bag is positioned to substantially coincide with an intended part of the leg of an individual such that the sold substance provides a therapeutic cooling to that area.

The '273 patent provides an overview of the prior cyrotherapy devices and that description will not be repeated here for the sake of brevity. In that description, however, it is noted that “the cooling effect of ice bags last an extended period of time, but typically bags do not contour to the body's curves for maximum application”.

What is thus desired is to provide an ice bag that more closely conforms to the knee area of an individual.

### SUMMARY OF THE INVENTION

The present invention provides a therapeutic ice bag that is shaped to conform closely to the knee of an individual. In particular, the ice bag has an essentially square shape and incorporates a timer so that the ice bag can be removed after a predetermined time period.

### DESCRIPTION OF THE DRAWINGS

For a better understanding of the present invention as well as other objects and further features thereof, reference is made to the following description which is to be read in conjunction with the accompanying drawing wherein:

FIG. 1 is a perspective view of the therapeutic ice system of the present invention;

FIG. 2 is a perspective view of the ice system with a timer embedded therein;

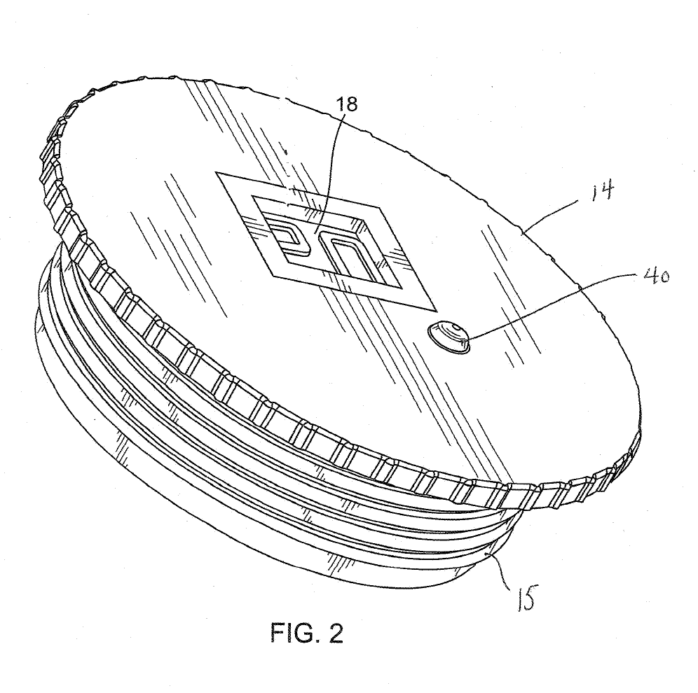

FIG. 3 illustrates the ice bag of FIG. 3 positioned on the knee of a user;

FIG. 4 is a top view illustrating the therapeutic ice system shown in FIG. 3 laid out to show the mechanism for securing the system to a user body part;

FIG. 5 is a circuit diagram of the timer controller;

FIG. 6 is a cross-section view of the stopper shown in FIG. 2; and

FIG. 7 is a bottom perspective view of the cap utilized in the therapeutic ice system of the present invention.

### DESCRIPTON OF THE INVENTION

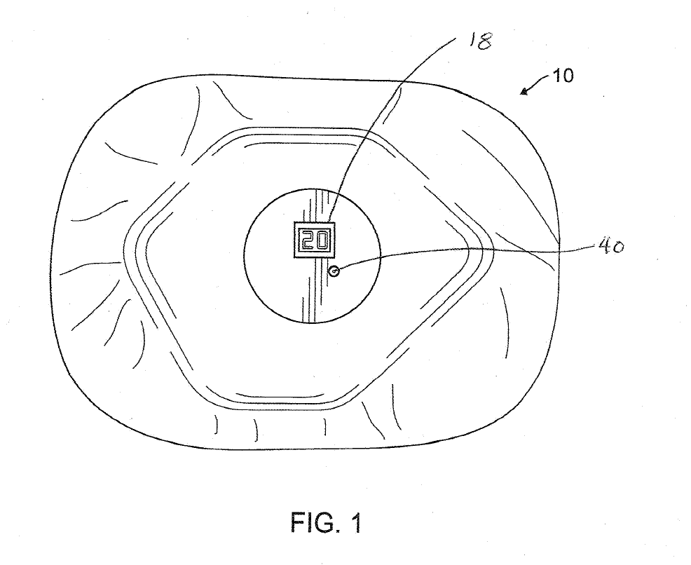

Referring to FIG. 1, a top plan view of the therapeutic ice bag 10 of the present invention is illustrated.

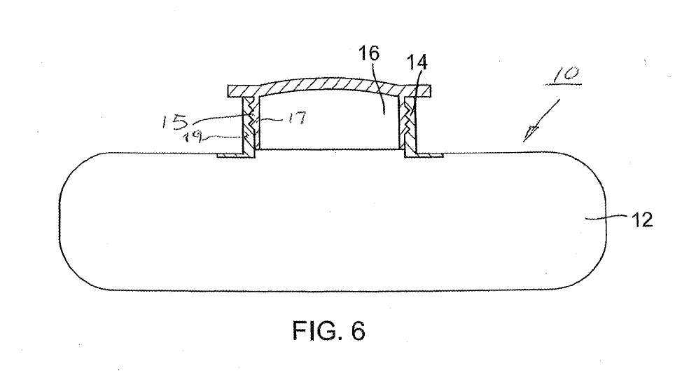

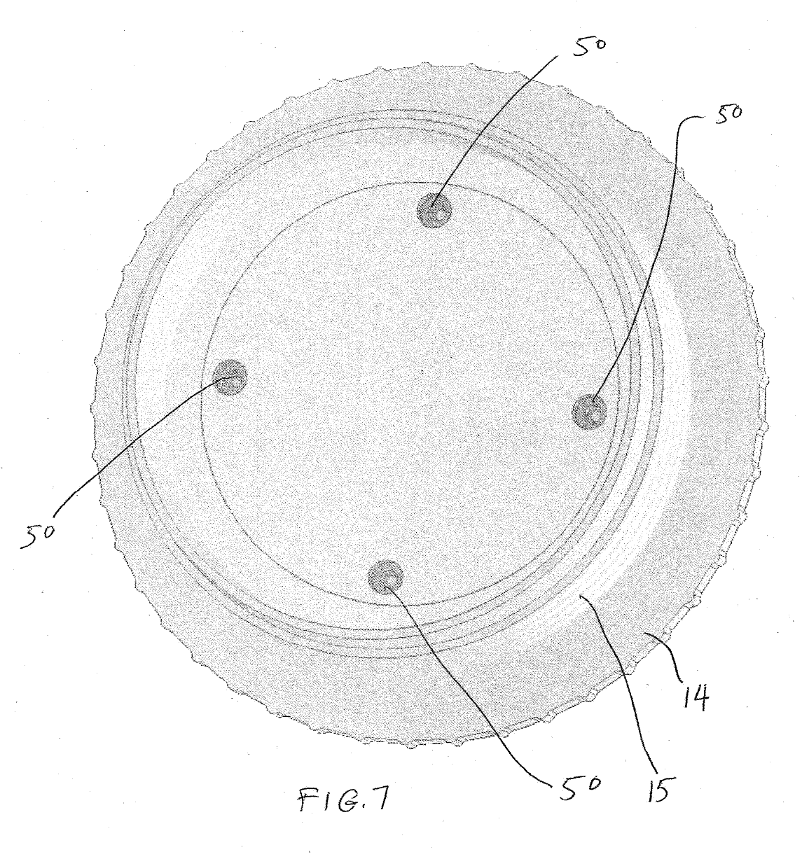

Ice bag 10 is preferably fabricated from elastic material, such as neoprene, and includes an internal receptacle area 12 (FIG. 6) having a cap 14 which covers the opening 16 to the receptacle area 12. Cap 14 has a threaded portion 15 which enables cap 14 to be secured to bag 10 via corresponding threads formed on bag extensions 19. A circuit device 18 is positioned in a receptacle area 20 and is used, among other functions, to keep track of the time that ice bag 10 has been worn by a user. LCD 21 provides a visual display of the timing sequences provided by device 18. As will be explained in more detail hereinafter, device 18 is preferably set for approximately 20 minutes to decrease inflammation and/or pain in the affected area by using ice. An audible warning is received when the 20 minute time has expired and the wearer knows to remove the ice bag 10 and replace, if necessary, at a predetermined time period after the initial application. This process can be repeated a number of times dependent upon the extent of the injury. Preferably, device 18 is positioned within cap 14 after molding of the cap and secured thereto by a plurality of screws and washers 50 as shown in FIG. 7.

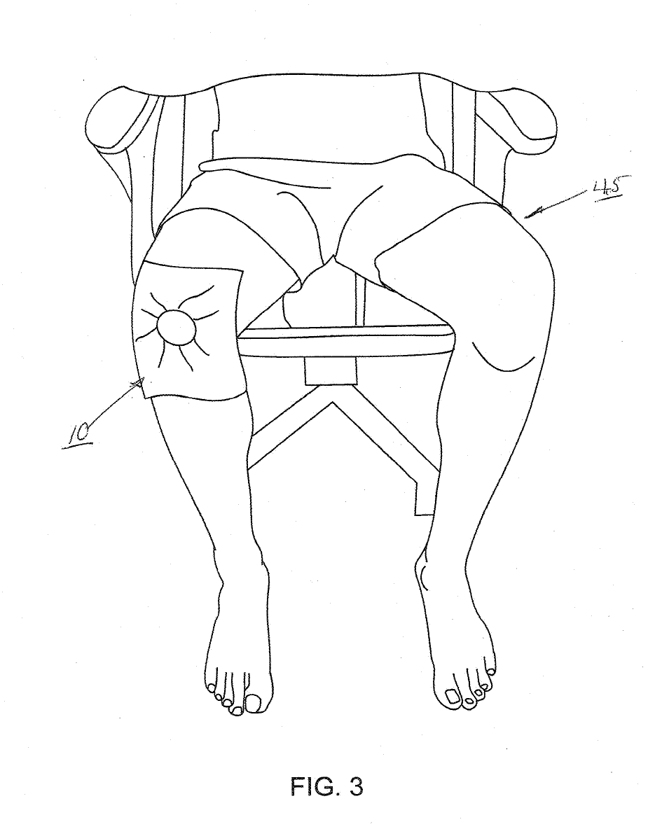

The shape of the ice bag 10 depends on what part of the body it will be applied to such as the knee, calf, hamstring, shoulder, back or foot. FIG. 3 illustrates ice pack 10 wrapped around the knee from side to side of a user 45.

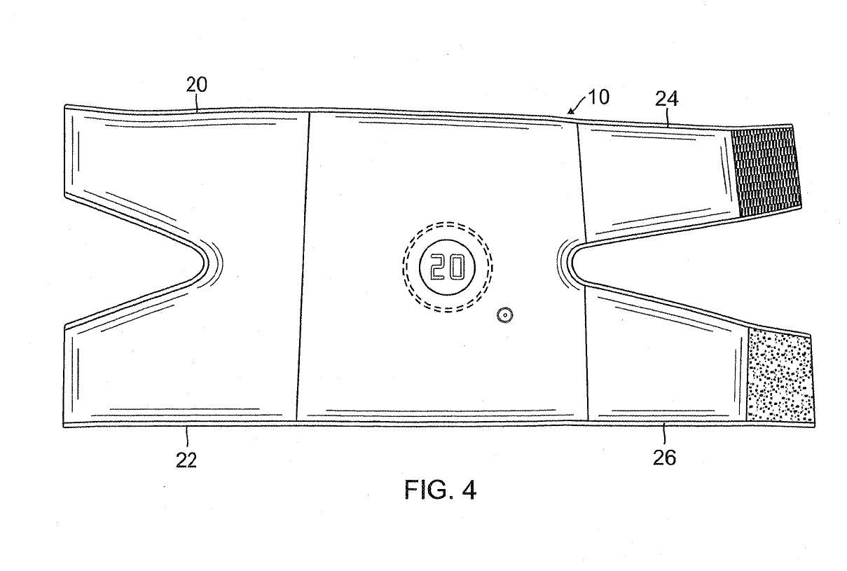

As shown in FIG. 4, ice bag 10 includes extensions 20, 22, 24 and 26 having Velcro® “hooks and loops” placed at the edges thereof, enabling bag 10 to be secured to the body part of a user, such as the knee.

The length A of the bag is approximately 12 inches, and the width B is approximately 9 inches.

In use, cap 14 is removed from bag 10 and cubed ice or crushed is inserted into receptacle area 12 (the diameter of the cap opening is preferably 2½ inches which allows for easy filling of the bag with ice cubes or crushed ice). The cap is then replaced and the timer 18 is set to 20 minutes, bag 10 then being secured to the user's knee as shown in FIG. 3. The LCD screen 21 readout, when not being used, is not illuminated (dark). The timer counts down from an initial twenty minute set point and provides an alarm when the countdown is completed. The power to the timer is turned off by pressing and holding button 40 down for at least 2 seconds. The LED screen 21 displays the number 20 when power is applied to the timer.

The timer operating method is as follows:

1. Button 40 is pressed illuminating LCD screen 21 with the numeral “20”;

2. Button 40 is then pressed once and an audio signal is heard and the countdown is initiated;

3. If the user decides to suspend the countdown, button 40 is pressed causing the numeral on the LCD screen 21 to flash: in order to restart the countdown, the user presses button 40;

4. When the count reaches zero, the timer circuit 18 causes a constant beep to be emitted;

5. To reset the timer, button 40 is pressed, the audio beeping sound ceases and the LCD screen 21 displays the numeral “20”.

If the timer circuit 18 idles for two minutes, or longer, it automatically turns off.

The bag 10 is reapplied to the knee after a predetermined time period and the process is repeated as often as necessary to reduce the knee swelling and inflammation

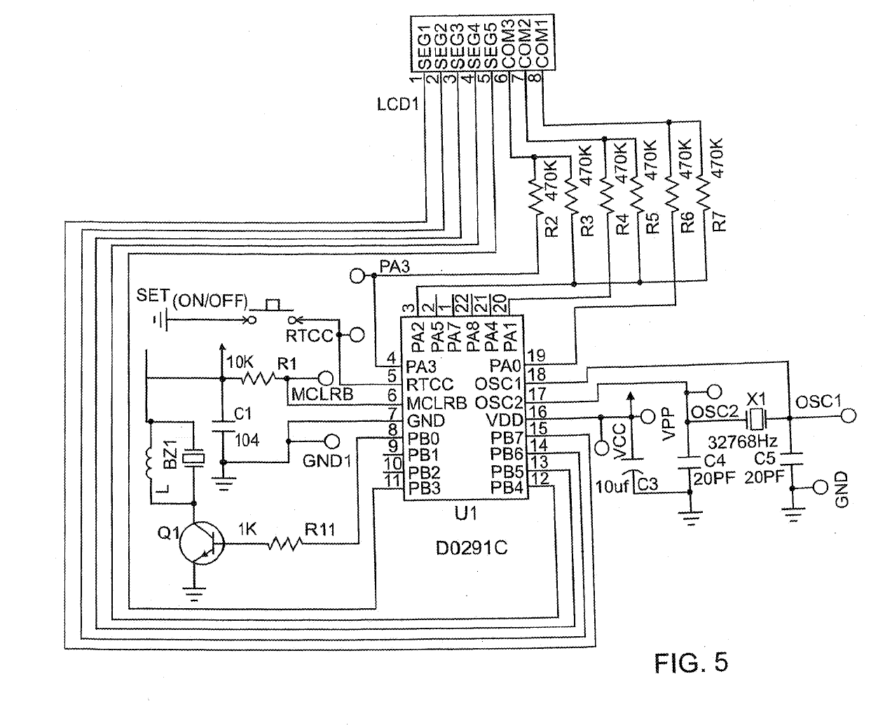

FIG. 5 is a schematic of the timer circuit used in timer device 18.

LCD1 represents the LCD display screen 21; resistors R2, R3, R4, R5, R6 and R7 are used to adjust screen 21; resistor R1 is the dormant pull-up component; C1 functions as the power decoupling capacitor; inductor L1, buzzer BZ1, NPN transistor Q1 and resistor R11 function as the buzzer alarm circuit; crystal oscillator X1 and capacitors C4 and C5 function as the oscillation circuit to generate timing/control signals; C3 is the power filter capacitor; and U1 is the circuit microprocessor controller.

While the invention has been described with reference to its preferred embodiments, it will be understood by those skilled in the art that various changes may be made and equivalents may be substituted for elements thereof without departing from the true spirit and scope of the invention. In addition, many modifications may be made to adapt a particular situation or material to the teachings of the invention without departing from its essential teachings.

What is claimed is:

1. A therapeutic cold pack for a body part comprising:
a shaped pad member having a predetermined length and width and having an internal area adapted to receive a cold substance, the pad member having first and second ends; and
a timer device secured to said shaped pad member.

2. The pad of claim 1 wherein said cold substance is ice.

3. The pad of claim 2 wherein extensions are formed on said first and second ends thereof.

4. The pad of claim 3 wherein attachment means are formed on said extension ends whereby the ends are securely coupled together when the cold pack is positioned on the body part of a user.

5. The pad of claim 4 wherein said body part is the knee.

6. The pad of claim 1 further including a cap for covering said internal area.

7. The pad of claim 6 wherein said timer is mounted to said cover.
